SEQUENCE LISTING (8 sequences)

SEQ ID NO: 1 - protein, 21 aa, Homo sapiens
QWAREIGAQLRRMADDLNAQY

SEQ ID NO: 2 - protein, 221 aa, Homo sapiens
MGHHHHHHHHSAGLVPRGSMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELY

SEQ ID NO: 3 - protein, 221 aa, Homo sapiens
MGHHHHHHHHSAGLVPRGSMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAVDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELY

SEQ ID NO: 4 - protein, 221 aa, homo sapiens
MGHHHHHHHHSAGLVPRGSMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDYFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELY

SEQ ID NO: 5 - protein, 221 aa, Homo sapiens
MGHHHHHHHHSAGLVPRGSMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDVFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELY

SEQ ID NO: 6 - protein, 221 aa, Homo sapiens
MGHHHHHHHHSAGLVPRGSMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDEFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELY

SEQ ID NO: 7 - protein, 221 aa, Homo sapiens
MGHHHHHHHHSAGLVPRGSMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGLFATVVEELFRDGVNWGRIVAFFEFGGVMCVESVNREMSPLVDNIALWMTEYLNRHLHTWIQDNGGWDAFVELY

SEQ ID NO: 8 - protein, 221 aa, Homo sapiens
MGHHHHHHHHSAGLVPRGSMAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFGEMSSQLHLTPFTARGRFATVVEELFRDGVNWGRIVAFFEFGGVMCVESVNREMSPLVDNIALWMTEYLNRHLHTWIQDNGGWDAFVELY

(19) United States
(12) Patent Application Publication
(10) Pub. No.: US 2022/0211718 A1
(43) Pub. Date: Jul. 7, 2022

(54) BCL-2 INHIBITORS FOR USE IN THE TREATMENT OF A BCL-2 MEDIATED CANCER CARRYING THE GLY101VAL MUTATION

(71) Applicants: LES LABORATOIRES SERVIER, SURESNES CEDEX (FR); NOVARTIS AG, Basel (CH)

(72) Inventors: James Brooke MURRAY, Linton (GB); Frèdéric COLLAND, Puiseux en France (FR); Audrey CLAPERON, Courbevoie (FR)

(73) Assignees: LES LABORATOIRES SERVIER, SURESNES CEDEX (FR); NOVARTIS AG, Basel (CH)

(21) Appl. No.: 17/609,874

(22) PCT Filed: May 11, 2020
(86) PCT No.: PCT/EP2020/063089

§ 371 Date: Nov. 9, 2021

Related U.S. Application Data
(60) Provisional application No. 62/847,477, filed on May 14, 2019.
(60) Provisional application No. 62/971,297, filed on Feb. 7, 2020.

(30) Foreign Application Priority Data
Jun. 7, 2019 (EP) 19178908.0

(51) Int. Cl. A61K 31/5377; A61P 35/02

(52) U.S. Cl. CPC A61K 31/5377; A61P 35/02

(57) ABSTRACT

The invention relates to a Bcl-2 inhibitor for use in the treatment of a Bcl-2 mediated cancer carrying at least 1, 2, 3, 4, 5 or all of the following mutations: (i) Gly101Val; (ii) Asp103Tyr; (iii) Asp103Val; (iv) Asp103Glu; (v) Arg129Leu and (vi) Ala113Gly; wherein the Bcl-2 inhibitor is N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide (Compound A) or 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-iH-pyrrole-3-carboxamide (Compound B), or a pharmaceutically acceptable salt thereof.

### FIELD OF THE INVENTION

The invention relates to a Bcl-2 inhibitor for use in the treatment of a Bcl-2 mediated cancer carrying at least 1, 2, 3, 4, 5 or all of the following mutations: (i) Gly101Val; (ii) Asp103Tyr; (iii) Asp103Val; (iv) Asp103Glu; (v) Arg129Leu and (vi) Ala113Gly; wherein the Bcl-2 inhibitor is N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide (Compound A, also known as S55746 or BCL201) or 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (Compound B), or a pharmaceutically acceptable salt thereof. The invention also relates to pharmaceutical composition comprising Compound A or Compound B for use in the treatment of Bcl-2 mediated cancer carrying at least 1, 2, 3, 4, 5 or all of the following mutations: (i) Gly101Val; (ii) Asp103Tyr; (iii) Asp103Val; (iv) Asp103Glu; (v) Arg129Leu and (vi) Ala113Gly. In a further embodiment, the Bcl-2 mediated cancer carrying at least 1, 2, 3, 4, 5 or all of the following mutations: (i) Gly101Val; (ii) Asp103Tyr; (iii) Asp103Val; (iv) Asp103Glu; (v) Arg129Leu and (vi) Ala113Gly is Chronic Lymphocytic Leukemia (CLL).

### BACKGROUND OF THE INVENTION

Apoptosis, or programmed cell death, is a physiological process that is crucial for embryonic development and maintenance of tissue homeostasis. Apoptotic-type cell death involves morphological changes such as condensation of the nucleus, DNA fragmentation and also biochemical phenomena such as the activation of caspases which cause damage to key structural components of the cell, so inducing its disassembly and death. Regulation of the process of apoptosis is complex and involves the activation or repression of several intracellular signalling pathways (Cory S. et al., Nature Review Cancer, 2002, 2, 647-656).

Deregulation of apoptosis is involved in certain pathologies. Increased apoptosis is associated with neurodegenerative diseases such as Parkinson's disease, Alzheimer's disease and ischaemia. Conversely, deficits in the implementation of apoptosis play a significant role in the development of cancers and their chemoresistance, in auto-immune diseases, inflammatory diseases and viral infections. Accordingly, absence of apoptosis is one of the phenotypic signatures of cancer (Hanahan D. et al., Cell 2000, 100, 57-70). The anti-apoptotic proteins of the Bcl-2 family are associated with numerous pathologies. The involvement of proteins of the Bcl-2 family is described in numerous types of cancer, such as colon cancer, breast cancer, small-cell lung cancer, non-small-cell lung cancer, bladder cancer, ovarian cancer, prostate cancer, chronic lymphoid leukaemia, follicular lymphoma, myeloma, and prostate cancer. Overexpression of the anti-apoptotic proteins of the Bcl-2 family is involved in tumorigenesis, in resistance to chemotherapy and in the clinical prognosis of patients affected by cancer. There is, therefore, a therapeutic need for compounds that inhibit the anti-apoptotic activity of the proteins of the Bcl-2 family.

Venetoclax (also known as ABT-199) is a selective Bcl-2 inhibitor that counteracts the interaction of Bcl-2 with BH3-only proteins thus inducing apoptosis. Venetoclax is approved (i) to treat adults with chronic lymphocytic leukemia (CLL) or small lymphocytic lymphoma (SLL), with or without 17p deletion, who have received at least one prior treatment and (ii) in combination with azacitidine, or decitabine, or low-dose cytarabine to treat adults with newly-diagnosed acute myeloid leukemia (AML) who are 75 years of age or older, or have other medical conditions that prevent the use of standard chemotherapy. However, a significant number of relapses are observed in CLL suggesting an acquired mechanism of resistance. In other high-affinity targeted therapies, specific mutations of the target have been demonstrated as responsible for resistance (Garraway & Janne, Cancer Discovery 2012, 2, 214-226). In different venetoclax-resistant derived leukemia and lymphoma cell lines, Bcl-2 mutations affecting its hydrophobic groove have been identified (Fresquet et al., Blood 2014, 123, 4111-4119; Tahir et al., BMC Cancer, 17:399). One mutation on Bcl-2 (Glycine substituted by a Valine in position 101: Gly101Val) was recently shown to be clinically relevant since identified in CLL samples from patients resistant to venetoclax. This Gly101Val mutation was associated with reduced venetoclax binding to the hydrophobic groove of Bcl-2 and resistance to venetoclax (Blombery et al., Cancer Discovery 2019, 9, 342-353). The Gly101 Val mutation may also provide a potential biomarker for impeding clinical relapse. Based on these clinical data, there is a need to identify new therapeutic agents that can be used to treat cancer patients who carry the Gly101Val mutation, and especially refractory and relapsed patients. More recently, other mutations in Bcl-2 such as Asp103Tyr (Aspartic acid substituted by a Tyrosine in position 103, also called D103Y), Asp103Val (Aspartic acid substituted by a Valine in position 103, also called D103V), Asp103Glu (Aspartic acid substituted by a Glutamic acid in position 103, also called D103E), Ala113Gly (Alanine substituted by a Glycine in position 113, also called A113G), Arg129Leu (Arginine substituted by a Leucine in position 129, also called R129L) and Val156Asp (Valine substituted by a Aspartic acid in position 156, also called V156D) have been identified in CLL patients treated with venetoclax while absent from naïve CLL patients (Tausch et al., Hematologica 2019, 9, e434-e437; Blombery et al., Blood 2020, 135(10), 773-777). Of note, genomic analysis demonstrates that one CLL patient could bear different Bcl-2 mutations (i.e. including G101V and D103Y). All mutations were observed to be present in different reads in NGS (Next Generation Sequencing) data, consistent with their presence in different leukemic cells (assuming heterozygosity) and with mutual exclusivity of the mutations (Blombery et al., Blood 2020, 135(10), 773-777).

There is a second generation of Bcl-2-specific inhibitors, including Compound A and Compound B, which have a partially overlapping but distinct Bcl-2 hydrophobic groove binding mode compared to venetoclax.

The chemical structure of Compound A (also known as S55746 or BCL201) is:

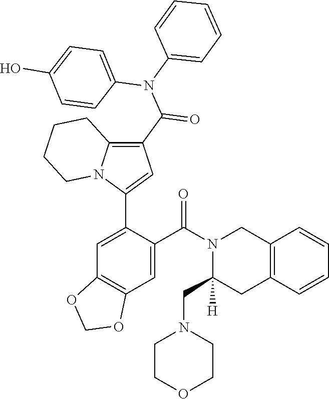 [SMILES: O=C(c1cc(-c2cc3c(cc2C(=O)N2Cc4ccccc4C[C@H]2CN2CCOCC2)OCO3)n2c1CCCC2)N(c1ccccc1)c1ccc(O)cc1]

The preparation of Compound A and its pharmacological effects in several cancer models are described in the literature (Casara et al., Oncotarget 2018. Vol. 9, No. 28, 20075-20088 and corresponding Supplementary Information). Moreover, Compound A and structurally-close analogues, their use as a Bcl-2 inhibitor for the treatment of cancer and pharmaceutical formulations thereof are also described in WO 2013/110890, the content of which is incorporated by reference. The preparation of Compound A is specifically disclosed in Example 1 of that document in the form of a hydrochloride salt.

Compound A occupies the region typically referred to as S1/2/3 in contrast to the venetoclax analogue (Souers c al., Nature Medicine 2013, 19, 202-208), which occupies a greater portion of the protein surface area including S2/3/4/5. Compound A forms a single hydrogen bond to the backbone carbonyl of residue A149 buried deep into S2. The size-independent enthalpic efficiency (0.83) for Compound A binding to Bcl-2 is suggestive of optimal polar and Van der Waal's interactions, indicative of highly specific binding (Casara et al., Oncotarget 2018, 9, 20075-20088).

The structure of Compound B is:

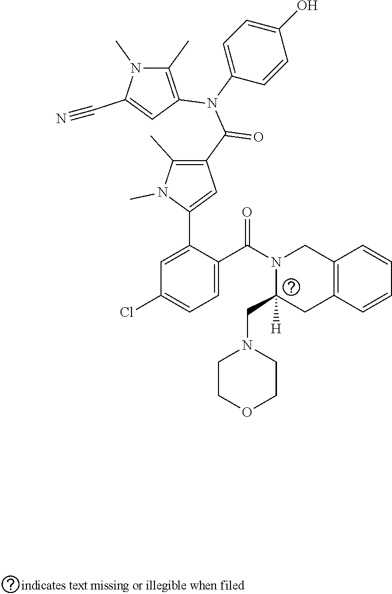 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C]

### 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide

The preparation of Compound B, its use as a Bcl-2 inhibitor for the treatment of cancer and pharmaceutical formulations thereof, are described in WO 2015/0111400, the content of which is incorporated by reference. Compound B is specifically disclosed in Example 386 of WO 2015/011400 in the form of a hydrochloride salt. It displays all the hallmarks of a Bcl-2 specific BH3-mimetic and exhibits robust antitumor activity in Bcl-2 dependent lymphoid tumor xenograft models while sparing platelets.

One assumption is that venetoclax on the one hand, and Compound A or Compound B on the other hand exhibit different binding modes. This structural hypothesis was confirmed by determining the different binding parameters of the three compounds in both wild-type Bcl-2 protein, Gly101Val, Asp103Tyr, Asp103Val, Asp103Glu, Arg129Leu and Ala113Gly mutant Bcl-2 proteins. The mutations in the Bcl-2 protein could thus be responsible for resistance to venetoclax but not to Compound A or Compound B. More particularly, Gly101Val mutation was located apart from the binding site of both Compound A and Compound B, in contrast to venetoclax. Furthermore, the Asp103Tyr mutation removes a key hydrogen bond between Bcl-2 and ABT-199 substantially reducing the affinity of ABT-199 for Bcl-2 harbouring the Asp103Tyr mutation. Compound B does not make any direct interaction with D103, it binds at least 9 angstroms away from D103, and thus its affinity for Bcl-2 harbouring the Asp103Tyr mutation is only modestly affected.

The Bcl-2 inhibitors according to the present invention have pro-apoptotic properties making it possible to use them in pathologies involving a defect in apoptosis, such as, for example, in the treatment of cancer and of immune and auto-immune diseases, and more specifically in patients resistant to venetoclax due to Bcl-2 mutations affecting its hydrophobic groove.

### SUMMARY

The present invention provides a Bcl-2 inhibitor for use in the treatment of Bcl-2 mediated cancer carrying at least 1, 2, 3, 4, 5 or all of the following mutations: (i) Gly101Val; (ii) Asp103Tyr; (iii) Asp103Val; (iv) Asp103Glu; (v) Arg129Leu and (vi) Ala113Gly; wherein the Bcl-2 inhibitor is Compound A, Compound B, or a pharmaceutically acceptable salt thereof. In particular, it has been found that the Bcl-2 inhibitors according to the invention have a strong activity for Bcl-2 Gly101Val mutant. The slight loss of activity observed between the wild type Bcl-2 protein and the Gly101Val mutant suggests that the administration of the Bcl-2 inhibitor according to the invention could induce a clinically relevant response in patients carrying the Gly101Val mutation. Additional cellular studies further confirmed that Compound B only showed a slight loss of potency (9 to 7 fold) in cell lines harboring the Bcl-2 Gly101Val mutant as compared to cell lines expressing the Bcl-2 wild-type protein. More generally, considering the biochemical profile disclosed herein, Compound B was found to display a better on-target activity on the set of mutants discussed above as compared to ABT-199. In addition to the biochemical data, cellular studies further demonstrated a shift of potency of 22-fold between ABT-199 and Compound B in cellular assays using KMS-12-PE cell lines overexpressing Bcl-2 Asp103Tyr. The present disclosure as a whole suggests that the Bcl-2 inhibitors according to the invention—and more especially Compound B—may have a beneficial effect in patients harbouring at least 1, 2, 3, 4, 5 or all of the following mutations: (i) Gly101Val; (ii) Asp103Tyr; (iii) Asp113Val; (iv) Asp103Glu; (v) Arg129Leu and (vi) Ala113Gly. In a particular aspect, CLL patients who have acquired one of the previous mutations further to venetoclax treatment are targeted.

In another aspect, the invention relates to a method for sensitizing a patient having a Bcl-2 mediated cancer carrying at least 1, 2, 3, 4, S or all of the following mutations: (i) Gly101Val; (ii) Asp103Tyr; (iii) Asp103Val; (iv) Asp103Glu; (v) Arg129Leu and (vi) Ala113Gly, who is (i) refractory to at least one anti-cancer agent, or (ii) in relapse after treatment with an anti-cancer agent, or both (i) and (ii), wherein the method comprises administering a therapeutically effective amount of a Bcl-2 inhibitor to said patient.

Overall, the invention described herein could enable to administrate a therapeutically effective amount of a composition including Compound A or Compound B to refractory or relapsed cancer patients, who carry at least 1, 2, 3, 4, 5 or all of the following mutations: (i) Gly101Val; (ii) Asp103Tyr; (iii) Asp113Val: (iv) Asp103Glu; (v) Arg129Leu and (vi) Ala113Gly; including venetoclax resistant patients.

### DETAILED DESCRIPTION OF THE INVENTION

‘Compound A’ means N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide.

‘Compound A, HCl’ means N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide in the form of a hydrochloride salt.

‘Compound B’ means 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide.

‘Compound B, HCl’ means that 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide is in the form of a hydrochloride salt.

‘Compound B. H₂SO₄’ means that 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide is in the form of a hydrogen sulfate salt.

‘Free molecule’ and ‘free base’ are used interchangeably herein and refer to Compound A or Compound B when not in salt form.

“Cancer” means a class of disease in which a group of cells display uncontrolled growth. Cancer types include haematological cancer (lymphoma and leukaemia) and solid tumors including carcinoma, sarcoma, or blastoma. In particular “cancer” refers to leukaemia, lymphoma or multiple myeloma, and more especially to chronic lymphocytic leukaemia, non Hodgkin lymphoma (including follicular lymphoma) or acute myeloid leukaemia.

‘Bcl-2 mediated cancer’ means a cancer in which the Bcl-2 protein can act as barrier to apopotosis and facilitate tumour development and resistance to cancer therapy. In particular, ‘Bcl-2 mediated cancer’ includes cancer characterized by a dysregulation of the Bcl-2 protein expression.

‘HP-β-cyclodextrin’ is also named ‘hydroxypropyl-β-cyclodextrin’ or ‘2-hydroxypropyl-β-cyclodextrin’ or ‘hydroxypropylbetadex’. In particular, the HP-β-cyclodextrin is marketed with the following product names: Cavitron™ W7HP7 (typical degree of substitution: 6.0-8.0; approximate molecular weight: 1520), Cavitron™ W7HP5 (typical degree of substitution: 4.1-5.1; approximate molecular weight: 1410), Kleptose™ HPB or Kleptose™ HP.

As used herein, the term ‘comprising’ means ‘including’, and is not intended to exclude the presence of any additional component, unless the context suggests otherwise, for example when the components together sum to 100%.

As used herein, the term ‘treat’, ‘treating’ or ‘treatment’ of any disease or disorder refers in one embodiment, to ameliorating the disease or disorder (i.e., slowing or arresting or reducing the development of the disease or at least one of the clinical symptoms thereof). In another embodiment, ‘treat’, ‘treating’ or ‘treatment’ refers to alleviating or ameliorating at least one physical parameter including those which may not be discernible by the patient. In yet another embodiment, ‘treat’, ‘treating’ or ‘treatment’ refers to modulating the disease or disorder, either physically, (e.g., stabilization of a discernible symptom), physiologically, (e.g., stabilization of a physical parameter), or both.

As used therein, a “therapeutically effective amount of the composition” means an effective amount of the composition according to the invention containing an effective dose of active principle to elicit a therapeutic benefit for the patient. For Compound A, the useful dosage ranges from 50 mg to 1500 mg per day expressed in terms of the free base. The dose of Compound B administered according to the invention is from 5 mg to 1000 mg expressed as free base.

As used therein, “a method for sensitizing” means a therapeutic method that allows ameliorating the disease or disorder (i.e., slowing or arresting or reducing the development of the disease or at least one of the clinical symptoms thereof) in relapsed or refractory patients. In a particular embodiment, “a method for sensitizing” means the restoration of a clinical response in patients resistant to an existing therapy.

As used therein, “targeted therapy” means a therapy that blocks the growth of cancer cells by interfering with specific targeted molecules needed for carcinogenesis and tumor growth, rather than by simply interfering with all rapidly dividing cells (as with traditional chemotherapy).

### EMBODIMENTS

Described below are a number of embodiments of the invention.

E1. A Bcl-2 inhibitor for use in the treatment of Bcl-2 mediated cancer carrying at least 1, 2, 3, 4, 5 or all of the following mutations: (i) Gly101Val; (ii) Asp103Tyr; (iii) Asp103Val; (iv) Asp103Glu; (v) Arg129Leu and (vi) Ala113Gly; wherein the Bcl-2 inhibitor is selected from the group consisting of N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide (Compound A) and 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (Compound B), or a pharmaceutically acceptable salt thereof.

E2. A Bcl-2 inhibitor for use in the treatment of Bcl-2 mediated cancer according to E1 wherein the cancer carries the Gly101Val mutation.

E3. A Bcl-2 inhibitor for use in the treatment of Bcl-2 mediated cancer according to E1 wherein the cancer carries the Asp103Tyr mutation.

E4. A Bcl-2 inhibitor for use in the treatment of Bcl-2 mediated cancer according to E1 wherein the cancer carries the Asp103Val mutation.

E5. A Bcl-2 inhibitor for use in the treatment of Bcl-2 mediated cancer according to E1 wherein the cancer carries the Asp103Glu mutation.

E6, A Bcl-2 inhibitor for use in the treatment of Bcl-2 mediated cancer according to E1 wherein the cancer carries the Arg129Leu mutation.

E7. A Bcl-2 inhibitor for use in the treatment of Bcl-2 mediated cancer according to E1 wherein the cancer carries the Ala113Gly mutation.

E8. A Bcl-2 inhibitor according to any of E1 to E7, wherein the Bcl-2 mediated cancer is chronic lymphocytic leukemia (CLL). Alternatively, the Bcl-2 mediated cancer is acute myeloid leukaemia (AML), multiple myeloma or non Hodgkin lymphoma.

E9. A Bcl-2 inhibitor according to any of E1 to E7, wherein the Compound A is in the form of a hydrochloride salt.

E10. A Bcl-2 inhibitor according to any of E1 to E7, wherein the Compound B is in the form of a hydrogen sulfate salt.

E11. A Bcl-2 inhibitor according to any of E1 to E7, wherein the Compound B is in the form of a hydrochloride salt.

E12. A Bcl-2 inhibitor according to any of E1 to E7, wherein Compound B is administered intravenously.

E13. A pharmaceutical composition comprising a Bcl-2 inhibitor according to any of E1 to E12 for use in the treatment of Bcl-2 mediated cancer carrying at least 1, 2, 3, 4, 5 or all of the following mutations: (i) Gly101Val; (ii) Asp103Tyr; (iii) Asp103Val; (iv) Asp103Glu; (v) Arg129Leu and (vi) Ala113Gly.

E14. A pharmaceutical composition comprising a Bcl-2 inhibitor according to E13 for use in the treatment of Bcl-2 mediated cancer carrying the Gly101Val mutation.

E15. A pharmaceutical composition comprising a Bcl-2 inhibitor according to E13 for use in the treatment of Bcl-2 mediated cancer carrying the Asp103Tyr mutation.

E16. A pharmaceutical composition comprising a Bcl-2 inhibitor according to E13 for use in the treatment of Bcl-2 mediated cancer carrying the Asp103Val mutation.

E17. A pharmaceutical composition comprising a Bcl-2 inhibitor according to E13 for use in the treatment of Bcl-2 mediated cancer carrying the Asp103Glu mutation.

E18. A pharmaceutical composition comprising a Bcl-2 inhibitor according to E13 for use in the treatment of Bcl-2 mediated cancer carrying the Arg129Leu mutation.

E19. A pharmaceutical composition comprising a Bcl-2 inhibitor according to E13 for use in the treatment of Bcl-2 mediated cancer carrying the Ala113Gly mutation.

E20. A method for sensitizing a patient having a Bcl-2 mediated cancer carrying at least 1, 2, 3, 4, 5 or all of the following mutations: (i) Gly101Val; (ii) Asp103Tyr; (iii) Asp103Val; (iv) Asp103Glu; (v) Arg129Leu and (vi) Ala113Gly;

who is (a) refractory to at least one anti-cancer agent, or (b) in relapse after treatment with an anti-cancer agent, or both (a) and (b), wherein the method comprises administering a therapeutically effective amount of a Bcl-2 inhibitor according to any of E1 to E12, to said patient.

E21. A method for sensitizing a patient having a Bcl-2 mediated cancer carrying the Gly101Val mutation who is (i) refractory to at least one anti-cancer agent, or (ii) in relapse after treatment with an anti-cancer agent, or both (i) and (ii), wherein the method comprises administering a therapeutically effective amount of a Bcl-2 inhibitor according to any of E1 to E12, to said patient.

E22. A method for sensitizing a patient having a Bcl-2 mediated cancer carrying the Asp103Tyr mutation who is (i) refractory to at least one anti-cancer agent, or (ii) in relapse after treatment with an anti-cancer agent, or both (i) and (ii), wherein the method comprises administering a therapeutically effective amount of a Bcl-2 inhibitor according to any of E1 to E12, to said patient.

E23. A method for sensitizing a patient having a Bcl-2 mediated cancer carrying the Asp103Val mutation who is (i) refractory to at least one anti-cancer agent, or (ii) in relapse after treatment with an anti-cancer agent, or both (i) and (ii), wherein the method comprises administering a therapeutically effective amount of a Bcl-2 inhibitor according to any of E1 to E12, to said patient.

E24. A method for sensitizing a patient having a Bcl-2 mediated cancer carrying the Asp103Glu mutation who is (i) refractory to at least one anti-cancer agent, or (ii) in relapse after treatment with an anti-cancer agent, or both (i) and (ii), wherein the method comprises administering a therapeutically effective amount of a Bcl-2 inhibitor according to any of E1 to E12, to said patient.

E25. A method for sensitizing a patient having a Bcl-2 mediated cancer carrying the Arg129Leu mutation who is (i) refractory to at least one anti-cancer agent, or (ii) in relapse after treatment with an anti-cancer agent, or both (i) and (ii), wherein the method comprises administering a therapeutically effective amount of a Bcl-2 inhibitor according to any of E1 to E12, to said patient.

E26. A method for sensitizing a patient having a Bcl-2 mediated cancer carrying the Ala113Gly mutation who is (i) refractory to at least one anti-cancer agent, or (ii) in relapse after treatment with an anti-cancer agent, or both (i) and (ii), wherein the method comprises administering a therapeutically effective amount of a Bcl-2 inhibitor according to any of E1 to E12, to said patient.

E27. A method according to any of E20 to E26 wherein the anti-cancer agent is a targeted therapy.

E28. A method according to E27 wherein the cancer is chronic lymphocytic leukemia (CLL).

E29. A method according to any of E27 or E28 wherein the targeted therapy is venetoclax (ABT-199).

E30. A method of treating a Bcl-2 mediated cancer in a patient, comprising the steps of:
(a) obtaining a biological sample from said patient and detecting whether the biological sample comprises at least 1, 2, 3, 4, 5 or all of the following mutations: (i) Gly101Val; (ii) Asp103Tyr; (iii) Asp103Val; (iv) Asp103Glu; (v) Arg129Leu and (vi) Ala113Gly;
(b) identifying said patients as having reduced likelihood of response to venetoclax;
(c) administering a therapeutically effective amount of a Bcl-2 inhibitor according to any of E1 to E12, to said patient based on the presence of the thus detected mutations.

E31. A method of treating a Bcl-2 mediated cancer in a patient, comprising the steps of:
(a) obtaining a biological sample from said patient and detecting whether the biological sample comprises the Gly101Val mutation;
(b) identifying said patients as having reduced likelihood of response to venetoclax;
(c) administering a therapeutically effective amount of a Bcl-2 inhibitor according to any of E1 to E12, to said patient based on the presence of the Gly101Val mutation.

E32. A method of treating a Bcl-2 mediated cancer in a patient, comprising the steps of:
(a) obtaining a biological sample from said patient and detecting whether the biological sample comprises the Asp103Tyr mutation;
(b) identifying said patients as having reduced likelihood of response to venetoclax;
(c) administering a therapeutically effective amount of a Bcl-2 inhibitor according to any of E1 to E12, to said patient based on the presence of the Asp103Tyr mutation.

E33. A method of treating a Bcl-2 mediated cancer in a patient, comprising the steps of:
(a) obtaining a biological sample from said patient and detecting whether the biological sample comprises the Asp103Val mutation;
(b) identifying said patients as having reduced likelihood of response to venetoclax;
(c) administering a therapeutically effective amount of a Bcl-2 inhibitor according to any of E1 to E12, to said patient based on the presence of the Asp103Val mutation.

E34. A method of treating a Bcl-2 mediated cancer in a patient, comprising the steps of:
(a) obtaining a biological sample from said patient and detecting whether the biological sample comprises the Asp103Glu mutation;
(b) identifying said patients as having reduced likelihood of response to venetoclax; 1(c) administering a therapeutically effective amount of a Bcl-2 inhibitor according to any of E1 to E12, to said patient based on the presence of the Asp103Glu mutation.

E35. A method of treating a Bcl-2 mediated cancer in a patient, comprising the steps of:
(a) obtaining a biological sample from said patient and detecting whether the biological sample comprises the Arg129Leu mutation;
(b) identifying said patients as having reduced likelihood of response to venetoclax;
(c) administering a therapeutically effective amount of a Bcl-2 inhibitor according to any of E1 to E12, to said patient based on the presence of the Arg129Leu mutation.

E36. A method of treating a Bcl-2 mediated cancer in a patient, comprising the steps of:
(a) obtaining a biological sample from said patient and detecting whether the biological sample comprises the Ala113Gly mutation;
(b) identifying said patients as having reduced likelihood of response to venetoclax; (c) administering a therapeutically effective amount of a Bcl-2 inhibitor according to any of E1 to E12, to said patient based on the presence of the Ala113Gly mutation.

### Example 1: Affinity Data of Compound A and Compound B on Bcl-2 Wild-Type and Bcl-2 Gly101Val Mutant

Fluorescence quenching assay measures the change fluorescence intensity of:
(i) C-terminally Cy5-labelled Bcl-2 wild-type protein (UniProtKB® primary accession number P10415) having an amino acid sequence (SEQ ID:02): [MGHHHHHHHHSAGLVPRGSMAHAGRTGYDNREIVMKYIHYKLSQRGY EWDAGDVGAAPPGAAPAPGIFSSQPGHTPHPAASRDPVARTSPLQTPAAP GAAAGPALSPVPPVVHLTLRQAGDDFSRRYRRDFAEMSSQLHLTPFTAR GRFATVVEELFRDGVNWGRIVAFFEFGGVMCVESVNREMSPLVDNIAL WMTEYLNRHLHTWIQDNGGWDAFVELY] which is linked at the C-terminus to the amino acid X which corresponds to a cysteine as defined below, or,
(ii) Bcl-2 Gly101Val mutant having an amino acid sequence (SEQ ID:03): [MGHHHHHHHHSAGLVPRGSMAHAGRTGYDNREIVMKYIHYKLSQRG YEWDAGDVGAAPPGAAPAPGIFSSQPGHTPHPAASRDPVARTSPLQTPA APGAAAGPALSPVPPVVHLTLRQAVDDFSRRYRRDFAEMSSQLHLTPFT ARGRFATVVEELFRDGVNWGRIVAFFEFGGVMCVESVNREMSPLVDNI ALWMTEYLNRHiLHTWIQDNGGWDAFVELY] which is linked at the C-terminus to the amino acid X which corresponds to a cysteine as defined below,
(where X=is cysteine labelled on the sulfur with sulpho-Cyanine5 from Lumiprobe GmbH catalogue number 13380)

upon binding of a C-terminally labelled peptide derived from PUMA (UniProtKB® primary accession number Q9BXHI) having an amino acid sequence (SEQ ID:01): [QWAREIGAQLRRMADDLNAQY] which is linked at the C-terminus to the amino acid X′, where X′ is cysteine labelled on the sulfur with TQ5WS from AAT Bioquest catalogue number 2079.

The addition of a compound which binds competitively to the same site as the peptide will result in an increase in the fluorescence intensity of the protein due to displacement of the fluorescence quencher.

An 11-point serial dilution of each compound was prepared in DMSO, the final buffer conditions were 10 mM 4-(2-hydroxyethyl)-1-piperazineethanesulfonic acid [HEPES], 150 mM NaCl, 0.05% Tween 20, pH 7.4 and 5% DMSO. The final protein concentration in the assay was 1 nM with the peptide present at 50 nM (Bcl-2 wild-type) or 100 nM (Bcl-2 Gly101Val). The experiments were incubated for 2 hours at room temperature before fluorescence intensity was measured on a Biotek SynergyNeo plate reader (Excitation 620 nm, emission 680 nm). The dose response curves were plotted with XL-Fit software using a 4-Parameter Logistic Model (Sigmoidal DoseResponse Model) and the inhibitory concentrations that gave a 50% increase in fluorescence intensity was determined (IC₅₀). The Kr values were determined from the IC₅₀ values according to Cer et al, Nucleic Acids Res, 2009, Jul. 1; 37(WebServer issue): W441-W445.

| | | Bcl-2 | |
|---|---|---|---|
| | Bcl-2 | Gly101Val | |
| | wild-type | mutant | |
| | cKi mean | cKi mean | Ratio Mutant/ |
| | (M) | (M) | Wild-type |
| ABT-199 | 1.1E−09 | 1.07E−07 | 97 |
| Compound A | 1.2E−09 | 2.37E−08 | 20 |
| Compound B | 1.0E−10 | 1.42E−09 | 14 |

The data demonstrate a significant loss of activity of ABT-199 on the Bcl-2 Gly101Val mutant as compared to the Bcl-2 wild-type protein, whilst the affinities of Compound A and Compound B are slightly affected by the mutation. Furthermore. Compound B is 75-fold more potent than ABT-199 on the Bcl-2 Gly101Val mutant.

### Example 2: In Vitro Cytotoxicity of Compound a and Compound B in Modified Cells Expressing Either Bcl-2 Wild-Type or Bcl-2 Gly101Val Mutant

Material and Methods

Cell lines were grown at 37° C. in a humidified atmosphere with 5% CO₂ in media recommended by the suppliers. RS4;11 (ATCC® CRL1873™) were purchased from American Type Culture Collection (ATCC) and KMS-12-PE (ACC 606) from the Leibniz-Institute DSMZ (Braunschweig, Germany). Lentiviral particles containing Bcl-2 wild-type (also named ‘Bcl-2 VT’) and Bcl-2 mutated on G101V (also named ‘Bcl-2 G101V’) were cloned into pcLV-CMV-DEST-IRES-TagRFP. Lentiviral particles (1×10⁶) were mixed with Polybrene at 8 μg/ml and transduced by spinoculation for 1 h at 32° C. and incubated overnight. After 8 days for KMS-12-PE and 21 days for RS4;11, TagRFP positive-cells were sorted by FACS. BCL2 expression was monitored by immunoblotting using anti-Flag and anti-BCL2 antibodies. Cells were seeded into 96-well plates and treated with 1:3.16 serial dilution of compounds (9 different concentrations). Cell viability was assessed using CellTiter-Glo® reagent following treatment with ABT-199, Compound A and Compound B for 72 h. Results were normalized to the viability of cells without compounds (control wells). The C₅₀ values were calculated using nonlinear regression algorithms in XCell software.

Results

| | KMS-12-PE Bcl-2 | KMS-12-PE Bcl-2 | Ratio |
|---|---|---|---|
| Compounds | WT (C50; nM) | G101V (C50; nM) | (G101V/WT) |
| Compound A | 57.3 | 961 | 17 |
| Compound B | 21 | 179 | 9 |
| ABT-199 | 22.5 | 931 | 41 |

| | RS4;11 Bcl-2 | RS4;11 Bcl-2 | Ratio |
|---|---|---|---|
| Compounds | WT (C50; nM) | G101V (C50; nM) | (G101V/WT) |
| Compound A | 82.3 | 495 | 6 |
| Compound B | 9.03 | 60.4 | 7 |
| ABT-199 | 10.7 | 130 | 12 |

### Conclusion

These results demonstrated a shirt of potency between ABT-199 and Compound Bin cellular assays using two different cell lines. Indeed, a significant loss of potency of ABT-199 was observed in cell lines overexpressing the Bcl-2 G101V mutant compared to the cell lines overexpressing the Bcl-2 wild-type protein (41 to 12-fold difference depending on cell lines). In contrast, Compound B showed only 9 to 7-fold difference between cell lines overexpressing either G101V Bcl-2 protein or wild type protein. While both Compound B and ABT-199 exhibited similar potency in both cell lines overexpressing wild type Bcl-2, Compound B showed 5 to 2-fold higher potency than ABT-199 in cell lines overexpressing the Bcl-2 G101V mutant.

### Example 3: Clinical Trial Protocol

A phase 1, open label, non-randomised, non-comparative, multi-center study, was set up to evaluate Compound B intravenously administered, in patients with Relapse or Refractory Acute Myeloid Leukaemia, Non Hodgkin Lymphoma, Multiple Myeloma or Chronic Lymphocytic Leukemia (CLL). In particular, the patients with CLL included in this study have relapsed or are refractory (except treatment failure, e.g. stable disease, non-response, progressive disease, death from any cause), as defined per iwCLL guidelines (Hallek M. et al, Blood, 2018, Vol. 131, 25, 2745-2760), from venetoclax treatment and without established alternative therapy. Approximately 60 patients will be enrolled in the study. This study is designed in two parts: part one for dose escalation, part two for dose expansion.

Primary Objectives:

Determine the safety profile (including Dose Limiting Toxicity (DLT) and Maximum Tolerated Dose (MTD(s)) and tolerability of Compound B in patients with Acute Myeloid Leukaemia (AML), Non Hodgkin Lymphoma (NHL), Multiple Myeloma (MM) or Chronic Lymphocytic Leukemia (CLL) and the recommended phase II dose (RP2D(s)) according to safety, PK and preliminary efficacy results.

Secondary Objectives:
To determine the pharmacokinetic (PK) profile of Compound B in plasma and in urine.
To assess the preliminary anti-tumor activity of Compound B using the appropriate response criteria for each evaluated population (AML, NHL, MM, CLL).

Exploratory Objectives Relative to CLL Patients with Mutations:
To assess the efficacy of Compound B on cells harboring Bcl-2 mutation(s), including the Gly101Val mutation and the Asp103Tyr mutation, by comparing pre- and on-treatment Bone Marrow Aspirate and blood samples in patients suffering from AML or CLL who previously received venetoclax. The mutations are detected by analyzing the patients samples using the Droplet Digital PCR technology (Vogelstein and Kinzler, Proc. Natl. Acad. Sci. USA 1999 96 Genetics; Olmedillas-López S et al, Mo Diagn Ther. 2017 October; 21(5):493-510).

Test Drug:
Compound B will be administered via i.v. infusion via a central or peripheral venous line.
Solution for infusion will be prepared using a 20 mL vials containing 150 mg of Compound B (expressed as free base) formulated with a HP-β-cyclodextrin as described below.
Duration of infusion, based on preliminary Safety and PK data, could be adapted.

Preparation of Lyophilisates of Compound B Solubilised in a HP-β-Cyclodextrin in 20 mL Vials:

The lyophilisates are prepared in 20 mL vials in which it will be possible to reconstitute the solution to be administered by the parenteral route. They are obtained by lyophilisation of a 20% Cavitron™ W7HP5 solution containing a dose of 20 mg/mL of Compound B (free base).

Procedure

In a 5 L reactor, weigh 1500 g of water. With magnetic stirring, create a vortex and then pour in 600 g of Cavitron™ W7HP5. Stir the medium at ambient temperature until the cyclodextrin is solubilised completely, and add 68.16 g of ‘Compound B, H₂SO₄’ and heat the solution to not more than 60° C. Place the suspension under magnetic stirring for several hours and then allow the medium to return to a temperature below 30° C. Measure the pH of the solution so obtained, then adjust it to pH 3.0 with 0,5M NaOH solution poured slowly. Make up the solution to a volume of 3 L by adding water, while maintaining magnetic stirring.

Pass the solution so obtained through a 0.2 μm filter.

Fill the 20 mL vials with the filtered solution so that each vial contains at least 150 mg of Compound B (expressed as free base) and subject the samples to a lyophilisation step.

The resulting lyophilisate is intended to be used for the preparation of a pharmaceutical composition for parenteral administration.

Dose Allocation Methodology:

A Bayesian Hierarchical Model (BHM), combined for all indications and guided by an escalation with overdose control (EWOC) method, will be used to guide dose escalation and estimate the MTD(s) based on the occurrence of DLT during Cycle 1.

Alternatively, an adaptive Bayesian Logistic Regression Model (BLRM) guided by an escalation with overdose control (EWOC) method, will be used to make dose recommendations based on the occurrence of DLT(s) during Cycle 1 and estimate the MTD(s)/RP2D(s) for the Compound B administered as a single agent.

Treatment Period:

The planned duration of treatment is until disease progression. Patients may be discontinued from treatment with the study drug earlier due to unacceptable toxicity and/or treatment is discontinued at the discretion of the investigator or the patient.

### Example 4: Affinity Data of Compound a and Compound B on Bcl-2 Wild-Type, Gly101Val Mutant, Bcl-2 Asp103Tyr Mutant, Asp103Val Mutant, Asp103Glu Mutant; Arg129Leu Mutant and Ala113Gly Mutant

Fluorescence quenching assay measures the change fluorescence intensity of:
(i) C-terminally Cy5-labelled Bcl-2 wild-type protein (UniProtKB® primary accession number P10415) having an amino acid sequence (SEQ ID:02): [MGHHHHHHHHSAGLVPRGSMAHAGRTGYDNREIVMKYIHYKLSQRG YEWDAGDVGAAPPGAAPAPGIFSSQPGHTPHPAASRDPVARTSPLQTPA APGAAAGPALSPVPPVVHLTLRQAVDDFSRRYRRDFAEMSSQLHLTPFT ARGRFATVVEELFRDGVNWGRIVAFFEFGGVMCVESVNREMSPLVDNI ALWMTEYLNRHiLHTWIQDNGGWDAFVELY] which is linked at the C-terminus to the amino acid X which corresponds to a cysteine as defined below, or,
(ii) Bcl-2 Gly101Val mutant having an amino acid sequence (SEQ ID:03): [MGHHHHHHHHSAGLVPRGSMAHAGRTGYDNREIVMKYIHYKLSQRG YEWDAGDVGAAPPGAAPAPGIFSSQPGHTPHPAASRDPVARTSPLQTPA APGAAAGPALSPVPPVVHLTLRQAVDDFSRRYRRDFAEMSSQLHLTPFT ARGRFATVVEELFRDGVNWGRIVAFFEFGGVMCVESVNREMSPLVDNI ALWMTEYLNRHiLHTWIQDNGGWDAFVELY] which is linked at the C-terminus to the amino acid X which corresponds to a cysteine as defined below, or,
(iii) Bcl-2 Asp103Tyr mutant having an amino acid sequence (SEQ ID:04): [MGHHHHHHHHSAGLVPRGSMAHAGRTGYDNREIVMKYIHYKLSQRG YEWDAGDVGAAPPGAAPAPGIFSSQPGHTPHPAASRDPVARTSPLQTPA APGAAAGPALSPVPPVVHLTLRQAVDDFSRRYRRDFAEMSSQLHLTPFT ARGRFATVVEELFRDGVNWGRIVAFFEFGGVMCVESVNREMSPLVDNI ALWMTEYLNRHiLHTWIQDNGGWDAFVELY] which is linked at the C-terminus to the amino acid X which corresponds to a cysteine as defined below, or,
(iv) Bcl-2 Asp103Val mutant having an amino acid sequence (SEQ ID:05): [MGHHHHHHHHSAGLVPRGSMAHAGRTGYDNREIVMKYIHYKLSQRG YEWDAGDVGAAPPGAAPAPGIFSSQPGHTPHPAASRDPVARTSPLQTPA APGAAAGPALSPVPPVVHLTLRQAVDDFSRRYRRDFAEMSSQLHLTPFT ARGRFATVVEELFRDGVNWGRIVAFFEFGGVMCVESVNREMSPLVDNI ALWMTEYLNRHiLHTWIQDNGGWDAFVELY] which is linked at the C-terminus to the amino acid X which corresponds to a cysteine as defined below, or,
(v) Bcl-2 Asp103Glu mutant having an amino acid sequence (SEQ ID:06): [MGHHHHHHHHSAGLVPRGSMAHAGRTGYDNREIVMKYIHYKLSQRG YEWDAGDVGAAPPGAAPAPGIFSSQPGHTPHPAASRDPVARTSPLQTPA APGAAAGPALSPVPPVVHLTLRQAVDDFSRRYRRDFAEMSSQLHLTPFT ARGRFATVVEELFRDGVNWGRIVAFFEFGGVMCVESVNREMSPLVDNI ALWMTEYLNRHiLHTWIQDNGGWDAFVELY] which is linked at the C-terminus to the amino acid X which corresponds to a cysteine as defined below, or,
(vi) Bcl-2 Arg129Leu mutant having an amino acid sequence (SEQ ID:07): [MGHHHHHHHHSAGLVPRGSMAHAGRTGYDNREIVMKYIHYKLSQRG YEWDAGDVGAAPPGAAPAPGIFSSQPGHTPHPAASRDPVARTSPLQTPA APGAAAGPALSPVPPVVHLTLRQAVDDFSRRYRRDFAEMSSQLHLTPFT ARGRFATVVEELFRDGVNWGRIVAFFEFGGVMCVESVNREMSPLVDNI ALWMTEYLNRHiLHTWIQDNGGWDAFVELY] which is linked at the C-terminus to the amino acid X which corresponds to a cysteine as defined below, or.
(vii) Bcl-2 Ala113Gly mutant having an amino acid sequence (SEQ ID:08): [MGHHHHHHHHSAGLVPRGSMAHAGRTGYDNREIVMKYIHYKLSQRG YEWDAGDVGAAPPGAAPAPGIFSSQPGHTPHPAASRDPVARTSPLQTPA APGAAAGPALSPVPPVVHLTLRQAVDDFSRRYRRDFAEMSSQLHLTPFT ARGRFATVVEELFRDGVNWGRIVAFFEFGGVMCVESVNREMSPLVDNI ALWMTEYLNRHiLHTWIQDNGGWDAFVELY] which is linked at the C-terminus to the amino acid X, (where X=is cysteine labelled on the sulfur with sulpho-Cyanine5 from Lumiprobe GmbH catalogue number 13380) upon binding of a C-terminally labelled peptide derived from PUMA (UniProtKB® primary accession number Q9BXHI) having an amino acid sequence (SEQ ID:01): [QWAREIGAQLRRMADDLNAQY] which is linked in C-terminal region to the amino acid X′, where X′ is cysteine labelled on the sulfur with TQ5WS from AAT Bioquest catalogue number 2079.

The addition of a compound which binds competitively to the same site as the peptide will result in an increase in the fluorescence intensity of the protein due to displacement of the fluorescence quencher.

The objective was to determine the K₁ of ABT-199, Compound A and Compound B as competitive binders of recombinant Bcl-2 wild type, G101V, D103Y, D103V, D103E, A113G, R129L mutants via PUMA quench reagent displacement, measured by fluorescence intensity.

The assays were carried out in black-walled, flat bottomed, low binding, 384-well plates. Compound (final conc. 5% DMSO) was mixed in buffer (10 mM 4-(2-hydroxyethyl)-1-piperazineethanesulfonic acid [HEPES], 150 mM NaCl, 0.05% Tween 20, pH 7.4), containing 50 nM of the peptide (probe), except in the case of G101V where 100 nM of the peptide was used, and [1 nM Bcl-2 wild type protein or 1 nM Bcl-2 mutant]. Assay plates were incubated about 2 hours at 18° C. and fluorescence intensity measured on a Synergy Neo reader (Ex. 620 nm, Em. 680 nm). The dose response curves were plotted with XL-Fit software using a 4-Parameter Logistic Model (Sigmoidal DoseResponse Model) and the inhibitory concentrations that gave a 50% increase in fluorescence intensity was determined (IC₅₀). The cK₁ values were determined from the IC₅₀ values according to Cer et al, Nucleic Acids Res, 2009, Jul. 1; 37(WebServer issue): W441-W445.

| | | Bel-2 | Bel-2 | Bel-2 | Bel-2 | Bel-2 | Bel-2 |
|---|---|---|---|---|---|---|---|
| | Bel-2 | Gly101Val | Asp103Tyr | Asp103Val | Asp103Glu | Arg129Leu | Ala113Gly |
| | wild-type | mutant | mutant | mutant | mutant | mutant | Mutant |
| | K₁ mean | K₁ mean | K₁ mean | K₁ mean | K₁ mean | K₁ mean | K₁ mean |
| | (M) | (M) | (M) | (M) | (M) | (M) | (M) |
| ABT-199 | 1.2E−09 | 9.8E−08 | inactive | 5.9E−08 | 1.7E−08 | 1.2E−08 | 4.1E−09 |
| | | | @1 μM | | | | |
| Compound A | 1.3E−09 | 2.3E−08 | 5.6E−09 | 3.7E−09 | 2.3E−09 | 3.3E−09 | 7.5E−09 |
| Compound B | 1.3E−10 | 1.9E−09 | 5.3E−10 | 2.8E−10 | 2.8E−10 | 1.0E−09 | 9.6E−10 |

For Compound A and Compound B, the data are inhibition constants (K₁) determined from complete binding inhibition curves (cK₁) whilst the data for ABT-199 are estimated from incomplete binding inhibition curves (eKi's) in most cases due to low activity (c: complete; e: estimated)

| | Bel-2 | | | | | | |
|---|---|---|---|---|---|---|---|
| | wild-type | Ratio | Ratio | Ratio | Ratio | Ratio | Ratio |
| | K₁ mean | Gly101Val/ | Asp103Tyr/ | Asp103Val/ | Asp103Glu/ | Arg129Leu/ | Ala113Gly/ |
| | (M) | Wild-type | Wild-type | Wild-type | Wild-type | Wild-type | Wild-type |
| ABT-199 | 1.2E−09 | 84 | N.C | 50 | 14 | 11 | 4 |
| Compound A | 1.3E−09 | 18 | 4 | 3 | 2 | 3 | 6 |
| Compound B | 1.3E−10 | 15 | 4 | 2 | 2 | 8 | 8 |

N.C: not calculated

The data demonstrate a significant loss of activity of ABT-199 on the Bcl-2 Asp103Tyr, Bel-2 Asp103Val and Bcl-2 Asp103Glu mutants as compared to the Bcl-2 wild-type protein, whilst the affinities of Compound A and Compound B are slightly affected by the mutations. The affinity of Compound B is moderately affected by the Arg129Leu and the Ala113Gly mutations. Despite this, Compound B is 12-fold and 4-fold more potent than ABT-199 on the Arg129Leu and the Ala113Gly mutants, respectively.

### Example 5: In Vitro Cytotoxicity of Compound B and ABT-199 in Modified Cells Expressing Asp103Tyr Mutant

Material and Methods

Cell lines were grown at 37° C. in a humidified atmosphere with 5% CO₂ in media recommended by the suppliers. KMS-12-PE (ACC 606) from the Leibniz-Institute DSMZ (Braunschweig, Germany). Lentiviral particles containing Bcl-2 mutated on D103Y were cloned into pcLV-CMV-DEST-IRES-TagRFP. Lentiviral particles (1×10⁶) were mixed with Polybrene at 8 μg/ml and transduced by spinoculation for 1 h at 32° C. and incubated overnight. TagRFP positive-KMS-12-PE cells were sorted by FACS after 8 days. Bcl-2 expression was monitored by immunoblotting using anti-Flag and anti-Bcl-2 antibodies. Cells were seeded into 96-well plates and treated at 9 points with 1:3.16 serial dilution of compounds. Cell viability was assessed using CellTiter-Glo® reagent following treatment with ABT-199 and Compound B for 72h. Results were normalized to the viability of cells without compounds (control wells). The C₅₀ values were calculated using nonlinear Regression algorithms in XCell software.

| | KMS-12-PE Bcl-2 |
|---|---|
| Compounds | D103Y (C50; nM) |
| Compound B | 83.7 |
| ABT-199 | 1830 |

### Conclusion

These results demonstrated a shift of potency between ABT-199 and Compound B in cellular assays using KMS-12-PE cell lines overexpressing BCL2 D103Y. Compound B showed about 22-fold higher potency than ABT-199 in cell lines overexpressing the Bcl-2 D103Y mutant.

Cell lines overexpressing other Bcl-2 clinical mutants including D103V, D103E, R129L and A113G mutants are currently being set up using same the same protocol to evaluate the compounds mentioned above.

Further in vivo experiments based on xenograft Models derived from modified cells expressing the Bcl-2 mutants (including the modified cell lines described in Examples 2 and 5) may show that Compound B could be an effective therapy for treating Bcl-2 mediated cancer carrying Bcl-2 mutations such as G101V and/or D103Y and/or others.

Additional results may be obtained by testing the efficacy of Compound Bin ex vivo samples from CLL patients carrying at least one of the Bcl-2 mutations selected from G101V, D103Y, D103V, D103E, R129L and A113G using a cell viability assay.

1-22. (canceled)

23. A method of treating a Bcl-2 mediated cancer carrying at least 1, 2, 3, 4, 5 or all of the following mutations: (i) Gly101Val; (ii) Asp103Tyr; (iii) Asp103Val; (iv) Asp103Glu; (v) Arg129Leu and (vi) Ala113Gly; in a subject in need thereof, comprising administering to the subject a therapeutically effective amount of of a Bcl-2 inhibitor selected from the group consisting of N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide (Compound A) and 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (Compound B), and pharmaceutically acceptable salts thereof, wherein the Bcl-2 inhibitor is administered alone or in combination with one or more pharmaceutically acceptable excipients.

24. The method according to claim 23, wherein the Bcl-2 mediated cancer carries the Gly101Val mutation.

25. The method according to claim 23, wherein the Bcl-2 mediated cancer carries the Asp103Tyr mutation.

26. The method according to claim 23, wherein the Bcl-2 mediated cancer carries the Asp103Val mutation.

27. The method according to claim 23, wherein the Bcl-2 mediated cancer carries the Asp103Glu mutation.

28. The method according claim 23, wherein the Bcl-2 mediated cancer is chronic lymphocytic leukemia (CLL).

29. The method according to claim 23, wherein the Compound A is in the form of a hydrochloride salt.

30. The method according to claim 23, wherein the Compound B is in the form of a hydrogen sulfate salt.

31. The method according to claim 23, wherein the Compound B is in the form of a hydrochloride salt.

32. The method according to claim 23, wherein Compound B is administered intravenously.

33. A method for sensitizing a patient having a Bcl-2 mediated cancer carrying at least 1, 2, 3, 4, 5 or all of the following mutations: (i) Gly101Val; (ii) Asp103Tyr; (iii) Asp103Val; (iv) Asp103Glu; (v) Arg129Leu and (vi) Ala113Gly;
who is (a) refractory to at least one anti-cancer agent, or (b) in relapse after treatment with an anti-cancer agent, or both (a) and (b), wherein the method comprises administering a therapeutically effective amount of a Bcl-2 inhibitor selected from the group consisting of N-(4-hydroxyphenyl)-3-{(6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide (Compound A) and 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1-pyrrol-3-yl)-N-(4-hydroxyphenyl)-1,2-dimethyl-1H-pyrrole-3-carboxamide (Compound B), and pharmaceutically acceptable salts thereof, to said patient.

34. A method for sensitizing a patient having a Bcl-2 mediated cancer carrying the Gly101Val mutation who is (i) refractory to at least one anti-cancer agent, or (ii) in relapse after treatment with an anti-cancer agent, or both (i) and (ii), wherein the method comprises administering a therapeutically effective amount of a Bcl-2 inhibitor selected from the group consisting of N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide (Compound A) and 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (Compound B), and pharmaceutically acceptable salts thereof, to said patient.

35. The method according to claim 33, wherein the anti-cancer agent is a targeted therapy.

36. The method according to claim 35, wherein the cancer is chronic lymphocytic leukemia (CLL).

37. The method according to claim 35, wherein the targeted therapy is venetoclax (ABT-199).

38. The method according to claim 34, wherein the anti-cancer agent is a targeted therapy.

39. The method according to claim 38, wherein the cancer is chronic lymphocytic leukemia (CLL).

40. The method according to claim 38, wherein the targeted therapy is venetoclax (ABT-199).

41. A method of treating a Bcl-2 mediated cancer in a patient, comprising the steps of:
(a) obtaining a biological sample from said patient and detecting whether the biological sample comprises at least 1, 2, 3, 4, 5 or all of the following mutations: (i) Gly101Val; (ii) Asp103Tyr, (iii) Asp103Val; (iv) Asp103Glu; (v) Arg129Leu and (vi) Ala113Gly;
(b) identifying said patients as having reduced likelihood of response to venetoclax;
(c) administering a therapeutically effective amount of a Bcl-2 inhibitor selected from the group consisting of N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide (Compound A) and 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (Compound B), and pharmaceutically acceptable salts thereof, to said patient based on the presence of the thus detected mutations.

42. A method of treating a Bcl-2 mediated cancer in a patient, comprising the steps of:
(a) obtaining a biological sample from said patient and detecting whether the biological sample comprises the Gly101Val mutation;
(b) identifying said patients as having reduced likelihood of response to venetoclax;
(c) administering a therapeutically effective amount of a Bcl-2 inhibitor selected from the group consisting of N-(4-hydroxyphenyl)-3-{6-[((3S)-3-(4-morpholinylmethyl)-3,4-dihydro-2(1H)-isoquinolinyl)carbonyl]-1,3-benzodioxol-5-yl-N}-phenyl-5,6,7,8-tetrahydro-1-indolizine carboxamide (Compound A) and 5-(5-chloro-2-{[(3)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (Compound B), and pharmaceutically acceptable salts thereof, to said patient based on the presence of the Gly101Val mutation.
